(19) United States
(12) Patent Application Publication
(10) Pub. No.: US 2007/0100205 A1
(43) Pub. Date: May 3, 2007

(54) IMAGE CAPTURING SYSTEM FOR ELECTRONIC ENDOSCOPE SYSTEM

(75) Inventor: Kenichi IRIYAMA, Tokyo (JP)

Correspondence Address: GREENBLUM & BERNSTEIN, P.L.C., 1950 ROLAND CLARKE PLACE, RESTON, VA, 20191, US

(73) Assignee: PENTAX CORPORATION, Tokyo (JP)

(21) Appl. No.: 11/553,188

(22) Filed: Oct. 26, 2006

(30) Foreign Application Priority Data
Oct. 27, 2005 (JP) P2005-312618

(51) Int. Cl. A61B 1/04; H04N 7/18

(52) U.S. Cl. 600/118; 600/109; 600/178; 348/68

(57) ABSTRACT

An image capturing system is provided with an illuminating unit configured to emit intermittent light that periodically illuminates an object, an image capturing element configured to capture an image of the object illuminated by the illuminating unit, and a control unit that controls the image capturing element to execute an image capturing operation. A period during which the object is periodically illuminated by the intermittent light includes an unstable starting period, a stable period and an unstable ending period, a timing at which the illuminating unit starts illuminating the object varying in the unstable starting period, a timing at which the illuminating unit stops illuminating the object varying in the unstable ending period. Further, the control unit controls the image capturing element to start and finish the image capturing operation within the stable period.

### BACKGROUND OF THE INVENTION

The present invention relates to an image capturing system for an electronic endoscope, and an electronic endoscope system employing such an image capturing system.

An electronic endoscope system typically includes an electronic endoscope provided with an image capturing element such as a CCD (Charge Coupled Device) for capturing images inside human cavity at a distal end of an inserting unit thereof, and a processor that receives and processes an image signal output by the image capturing element to generate a video signal, which is transmitted to a monitor. Such an electronic endoscope system is configured such that light output by a light source provided in the processor is transmitted, using an optical fiber and the like inserted through the electronic endoscope, to a target portion inside the human cavity from the distal end of the electronic endoscope, and images of the illuminated portion are captured consecutively by an image capturing element (e.g., CCD). The image capturing element outputs image signals representing the captured images, which are processed by the processor and displayed by the monitor as an animated or still image.

Conventionally, in order to adjust the brightness of the images, a so-called electronic shutter is employed. That is, a time period in which the CCD accumulates the charges is controlled to adjust the brightness of the displayed image. In such a system, the light is continuously turned ON to illuminate the target portion continuously, and a time period for accumulating the charges for each frame of image is adjusted using the electronic shutter technique.

In the electronic endoscope system described above, however, since the light is continuously turned ON, the temperature of the tip portion of the electronic endoscope may increase excessively. Since the distal end portion is inside the human cavity, it is desirable that the temperature of the distal end portion of the electronic endoscope does not increase, particularly when it is inside the human cavity.

In order to avoid such a problem, there exists an electronic endoscope system employing a so-called chopper mechanism. An example of such an electronic endoscope system is disclosed in U.S. Pat. No. 4,710,807 (hereinafter, referred to as '807 patent. In '807 patent, using the chopper mechanism, intermittent light is transmitted from the light source to the light guide extending through the endoscope, and the intermittent light is emitted to the target portions. With this configuration, since the intermittent light is used, the temperature of the distal end portion of the endoscope will not increase.

According to '807 patent, however, since the chopper mechanism employs a mechanically rotating member, a so-called rotational jitter may occur in ON/OFF timings of the light illuminating the target portion. Because of the rotational jitter, an exposure period for each frame may vary. That is, the brightness of the consecutively captured image frames varies frequently, and the video image displayed on the monitor may flicker and is annoying to observe.

### SUMMARY OF THE INVENTION

Aspects of the invention are advantageous in that an electronic endoscope is capable of capturing images having less variation in brightness without causing the distal end portion of the endoscope to be unintentionally heated.

According to an aspect of the invention, there is provided an image capturing system, which is provided with an illuminating unit configured to emit intermittent light that periodically illuminates an object, an image capturing element configured to capture an image of the object illuminated by the illuminating unit, and a control unit that controls the image capturing element to execute an image capturing operation, which includes a start of exposure and completion of accumulating electrical charges in the image capturing element. A period during which the object is periodically illuminated by the intermittent light includes an unstable starting period, a stable period and an unstable ending period, a timing at which the illuminating unit starts illuminating the object varying in the unstable starting period, a timing at which the illuminating unit stops illuminating the object varying in the unstable ending period. Further, the control unit controls the image capturing element to execute the image capturing operation only within the stable period.

According to another aspect of the invention, there is provided an electronic endoscope system including an electronic endoscope and a processor, which is further provided with an illuminating unit provided to the processor and configured to emit intermittent light that is guided inside the electronic endoscope and emitted from a tip end of the electronic endoscope to periodically illuminate an object, an image capturing element provided to the endoscope and configured to capture an image of the object illuminated by the illuminating unit, and a control unit provided to the endoscope and configured to control the image capturing element to execute an image capturing operation, which includes the start of exposure and the completion of accumulating the electrical charges in the image capturing element. A period during which the object is periodically illuminated by the intermittent light includes an unstable starting period, a stable period and an unstable ending period, a timing at which the illuminating unit starts illuminating the object varying in the unstable starting period, a timing at which the illuminating unit stops illuminating the object varying in the unstable ending period. Further, the control unit controls the image capturing element to execute the image capturing operation only within the stable period.

The control unit may be configured to transmit a sweep pulse to the image capturing element after the unstable start period has elapsed and a charge transfer pulse to the image capturing element before the unstable end period begins.

The control unit may be configured to change at least one of a timing at which the sweep pulse is transmitted to the image capturing element and a timing at which the charge transfer pulse is transmitted to the image capturing element to vary a charge accumulating period.

The time at which the sweep pulse is transmitted to the image capturing element may be variable.

The time at which the charge transfer pulse is transmitted to the image capturing element may be set to a timing immediately before the beginning of the unstable end period.

The illuminating unit may be provided with a light source emitting continuous light and a rotational chopper mechanism having a rotatable member formed with a light shielding portion that shields the light emitted by the light source and a light transmitting portion that allows the light emitted by the light source to pass through.

The rotational chopper mechanism may be provided with a motor which is controlled in accordance with a phase locked loop method using a signal synchronized with the charge transfer pulse.

### BRIEF DESCRIPTION OF THE ACCOMPANYING DRAWINGS

FIG. 1 is a block diagram showing a configuration of an electronic endoscope system according to aspects of the invention.

FIG. 2 is a front view of a rotary disk according to aspects of the invention.

FIGS. 3A-3F show a timing chart illustrating an image capturing operation according to aspects of the invention.

FIGS. 4A-4F show a timing chart illustrating an image capturing operation according to a comparative electronic endoscope system.

### DETAILED DESCRIPTION OF THE EMBODIMENTS

Hereinafter, referring to the accompanying drawings, an electronic endoscope system including an image capturing system according to an embodiment of the invention will be described in detail.

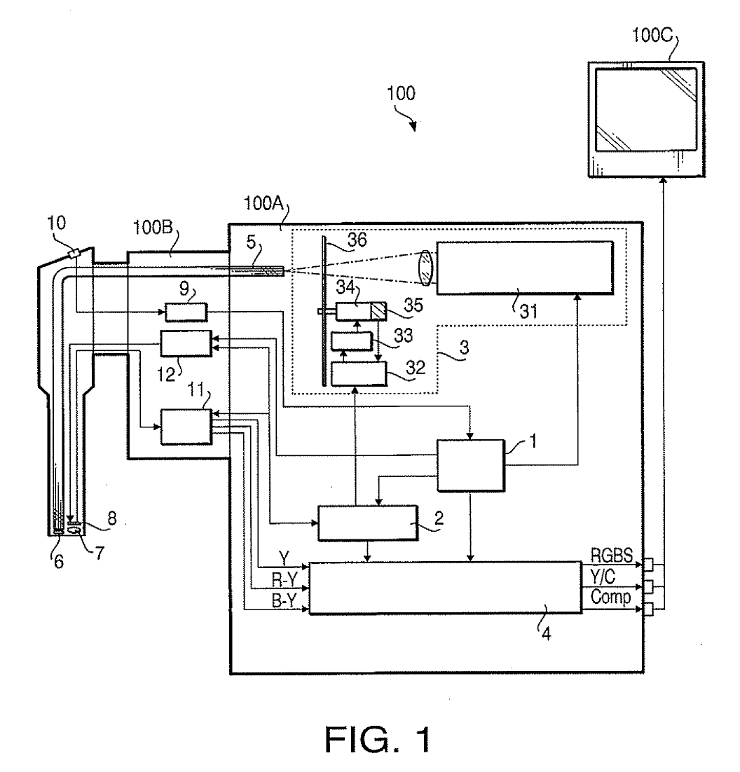

FIG. 1 is a block diagram showing a configuration of an electronic endoscope system 100 according to an embodiment of the invention. The electronic endoscope 100 is used for observation and/or diagnosis inside a human cavity of a patient. The electronic endoscope system 100 includes a processor 100A, an electronic endoscope 100B having an inserting section to be inserted in the human cavity, and a monitor 100C.

The processor 100A is provided with a system controller 1, a timing controller 2, a light source unit 3 and an image processing unit 4. The system controller 1 and the timing controller 2 operate to control an entire electronic endoscope system 100. The light source unit 3 is configured to generate intermittent light for illuminating target portions inside the human cavity when images of the target portions are to be captured using the electronic endoscope 100B. The image processing unit 4 applies a predetermined processing to an image signal which is output by the electronic endoscope 100B when the image of the target portion is captured. The image processing unit 4 outputs the processed image signal, which is input in the monitor 100C for display.

The inserting section of the electronic endoscope 100B includes a flexible tube. Inside the electronic endoscope, a light guide 5 formed of a bundle of optical fibers extends throughout the electronic endoscope 100B. The light emitted by the light source 31 is transmitted through the light guide 5. At the tip end of the light guide 5, a light distribution lens 6 is provided. The light passed through the light guide 5 is emerged from the tip end surface thereof, and is incident on the light distribution lens 6. As shown in FIG. 1, at the distal end of the electronic endoscope 100B, another lens (i.e., an objective lens 7) is provided. On the rear side (i.e., proximal end side) of the objective lens 7, a solid state image capturing element 8 is arranged. Further, the electronic endoscope 100B is provided with a scope control unit 9, a switch 10, a DSP (Digital Signal Processor) 11 and a CCD driving circuit 12, functions of which will be described later.

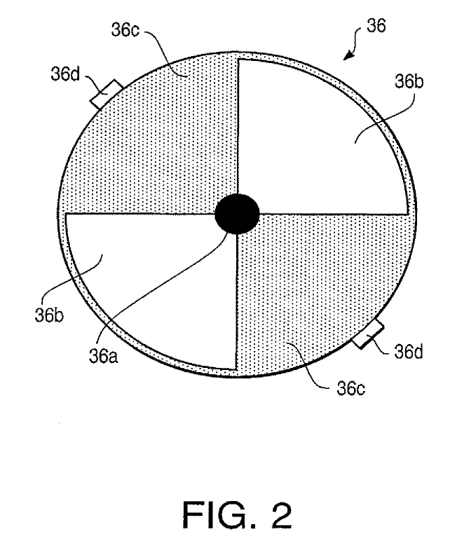

Under control of the system controller 1, the light source 31 of the light source unit 3 emits continuous light. On the optical path of the continuous light emitted by the light source 31, a rotary disk 36 is inserted. FIG. 2 shows a front view of the rotary disk 36. The rotary disk 36 is a disk-shaped member made of opaque material (light shielding material), and arranged to be rotatable about a rotary shaft 36a. The rotary disk 36 is formed with openings 36b and 36b, and light-shielding areas 36c and 36c, each has the same central angle. Further, at the peripheral end of the light shielding areas 36c and 36c, projected portions 36d and 36d are formed, respectively.

The rotary disk 36 is rotated by a motor 34, which is driven to rotate under control of a motor driving circuit 32 via a driver 33. The motor 34 is connected with a rotary incremental encoder 35. The rotary incremental encoder 35 has a sensor unit (not shown) which detects passage of each of the projected portions 36d and 36d of the rotary disk 36 and outputs a pulse. According to the embodiment, the motor control circuit 32 compares the pulses periodically output from the rotary incremental encoder 35 with a synchronizing signal output by the timing controller 2. The synchronizing signal is output at the same timing of a vertical transfer pulse (described later). The motor control circuit 32 executes a PLL (Phase Locked Loop) control in order to control the rotation of the rotary disk 36 so that a phase difference between the synchronizing signal and the pulses output by the rotary incremental encoder 35 is cancelled.

The continuous light emitted by the light source 31 is incident on one end of the light guide 5 only when the opening 36b is located at the optical path (i.e., only when the light is allowed to pass through the rotary disk 36). Since the rotary disk 36 is rotated, the continuous light emitted by the light source 31 is converted into intermittent light by the function as a chopper of the rotary disk 36, and is intermittently incident on the light guide 5.

The light is transmitted through the light guide 5, emerged from the other end of the light guide 5, passed through the distribution lens 6, and emerged from the distal end of the flexible tube of the insertion section toward the target portion. The light is then reflected by in vivo tissues inside the human cavity and incident on (i.e., converged on) the solid state image capturing element 8 via the objective lens 7. The solid state image capturing element 8 accumulates electric charges in accordance with the quantity of light incident thereon. Specifically, the system controller 1 controls the driving circuit 12 to periodically output a sweep pulse and transfer pulses so that electrical charges corresponding to the image formed on the solid state image capturing element 8 are accumulated and transferred to the DSP 11 as an image signal. When the sweep pulse is input to the image capturing element 8, the accumulated charges are swept away (i.e., the charges are not used for generating the image signal), and an accumulation process is restarted. When the transfer pulses are input, the accumulated charges are consecutively output to the DSP 11 as the image signal.

The DSP 11 applies predetermined processing to the image signal received from the image capturing element 8 to modify the image signal suitable to the processor 100A. The predetermined processing may include, for example, a clipping process for limiting a dynamic range of the image signal within a certain range and a gamma compensation process for compensating for a gamma characteristic so that brightness/color-reproducibility characteristics are suitable for the monitor 100C.

The image signal output by the solid state image capturing element 8 is sampled by the DSP 11 and converted into a brightness component signal Y and color difference signals R-Y and B-Y; which are transmitted to the processor 100A.

The signals (i.e., Y signal, R-Y signal and B-Y signal) output by the DSP 11 are processed by the image processing unit 4 of the processor 100A and a video signal suitable to the monitor 100C is generated and output. The monitor 100C displays an image according to the video signal output by the image processing unit 4.

Next, an image capturing process according to aspects of the invention will be described in detail. The image capturing process is executed when the operator operates the switch 10. Specifically, in response to the operation of the switch 10, the scope control unit 9 outputs a control signal, which is transmitted in the system controller 1. Then, the system controller 1 starts the image capturing process.

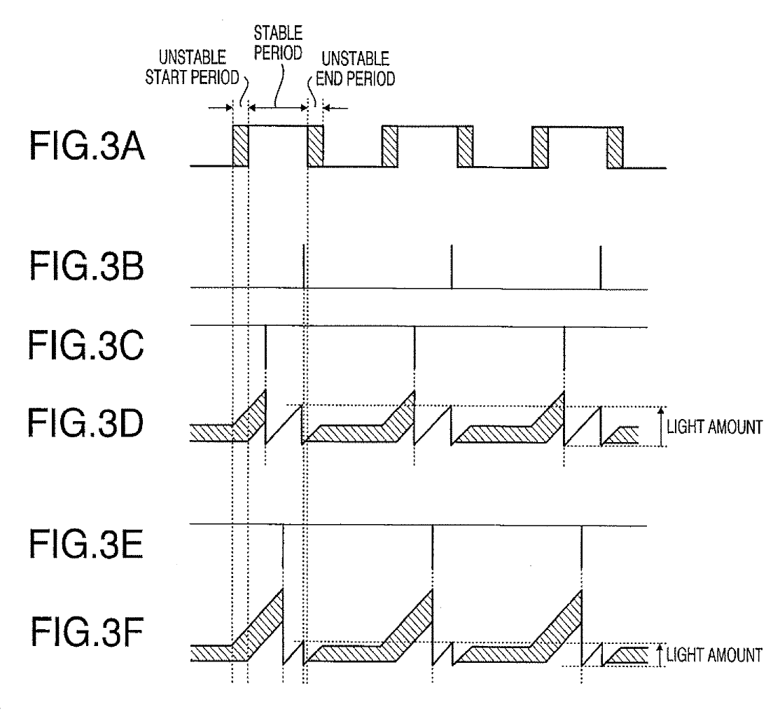

FIGS. 3A-3F show a timing chart illustrating the image capturing process. FIG. 3A shows light emission timings of the intermittent light emitted by the light source unit 3. Generally, when the intermittent light is generated using the rotary disk 36, due to a rotational jitter of the motor 34, there exist an instability in a timing when the light is turned ON and a timing when the light is turned OFF. That is, the timing when the light is turned ON may vary with respect to a designed timing, so is the timing when the light is turned OFF. Each of hatched areas in FIG. 3A represent a range within which the timing may vary due to the rotation jitter of the motor 34. Hereinafter, each of these areas will be referred to as an unstable period. The light may be turned ON or turned OFF during the unstable period. That is, a period during which the light is turned ON and stays turned ON includes an unstable starting period, a stable period and an unstable ending period. A timing at which the light is turned ON varies in the unstable starting period, and a timing at which the light is turned OFF varies in the unstable ending period. In contrast, areas between the unstable periods and the light stays turned ON can be regarded as periods where the light should be always turned ON, which periods will be referred to as stable periods. Because of the existence of the unstable periods, light emitting periods of the intermittent light may vary.

As previously mentioned, the variation of the light emitting periods is mainly caused by the rotational jitter of the motor 34. According to the embodiment, therefore, the degree of the jitter is measured in advance. Then, based on the measurement result, the stable period is defined for each light emitting period, which is set to the system controller 1.

The system controller 1 transmits a vertical transfer pulse to the solid state image capturing element 8 periodically via the timing controller 2 and the driver 12. FIG. 3B shows the vertical transfer pulse transmitting timing. As shown in FIG. 3B, the system controller 1 transmits the vertical transfer pulse before the end of the stable period. With this control, it becomes possible to make maximum use of the intermittent light (during the stable period).

The system controller 1 also transmits a charge sweep pulse to the solid state image capturing element 8 periodically via the timing controller 2 and the driver 12. FIG. 3C shows the charge sweep pulse transmitting timing when an electronic shutter speed is set to its initial speed (which is a medium speed). As shown in FIG. 3C, the system controller 1 transmits the charge sweep pulse after the beginning of the stable period and before the vertical transfer pulse within the same stable period.

When receiving the charge sweep pulse, the solid state image capturing element 8 sweeps away the charges accumulated immediately before receipt of the charge sweep pulse, When receiving the vertical transfer pulse as the charge transfer pulse, the solid state image capturing element 8 transfers the charges accumulated immediately before receipt of the vertical transfer pulse to the DSP 11 as the image signal. Thus, in either case (when the image capturing element 8 receives the pulse), the charge-accumulated status is reset.

FIG. 3D shows a change of the accumulated received light amount (accumulated electric charges) of the solid state image capturing element 8 controlled as above. In a graph shown in FIG. 3D, the hatched areas indicate light amount variable areas due to the unstable periods of the intermittent light. That is, due to the unstable periods, the accumulated light amount on the solid state image capturing element 8 varies within the hatched areas in FIG. 3D.

According to the embodiment, since the charge sweep pulse and the vertical transfer pulse are applied in a manner described above, regardless of the existence of the unstable periods due to the rotational jitter of the motor 34 or the like, the accumulated light amount contributing to generation of the image signal is not affected by the unstable periods. Thus, the image can be captured at a desired shutter speed (i.e., the speed of the electronic shutter).

Further, since the intermittent light is used, unnecessary increase of the temperature at the tip of the flexible tube as in the conventional art can be prevented.

FIG. 3E shows the charge sweep pulse when the speed of the electronic shutter is to be increased (i.e., the exposure period is shortened). According to the embodiment, the timing of the vertical transfer pulse is fixed at a timing immediately before the end of the stable period. In order to obtain a higher shutter speed, as shown in FIG. 3E, the timing of the charge sweep pulse is delayed.

FIG. 3F shows a change of the accumulated received light amount (accumulated electric charges) of the solid state image capturing element 8 when the charge sweep pulses shown in FIG. 3E are used. As understood from FIGS. 3E and 3F, regardless of the existence of the unstable periods, the accumulated light amount contributing to generation of the image signal is not effected by the unstable periods.

It should be noted that, in the embodiment described above, the timing of the vertical transfer pulse in each stable period is fixed and the timing of the charge sweep pulse is changed to vary the electronic shutter speed. The invention needs not be limited to such a configuration. For example, both the timings of the vertical transfer pulse and the charge sweep pulse within the stable period can be changed, or the timing of the charge sweep pulse is fixed at a timing immediately after the beginning of the stable period, and the timing of the vertical transfer pulse within the stable period may be changed.

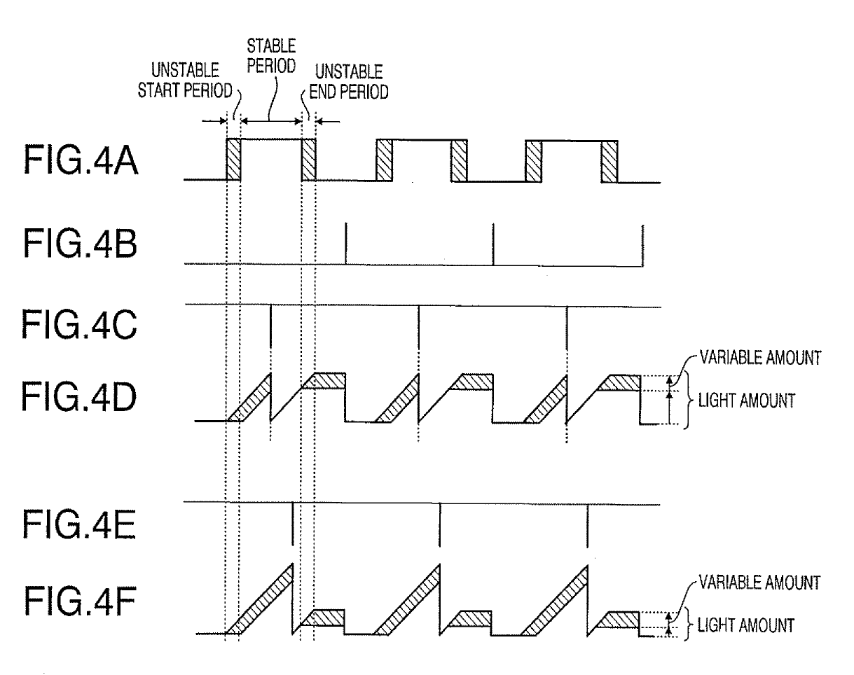

FIGS. 4A-4F show a timing chart of a comparative example, in which the transfer pulses are output after the light is turned OFF (see FIG. 4B). FIG. 4D or 4F shows the change of the accumulated light amount. In this example, a variable amount corresponding to the unstable periods (see FIG. 4A) is included. As understood from FIGS. 4D and 4F, the light amount when the vertical transfer pulse includes a variable component (hatched portion). That is, the light amount value at the vertical transfer pulse is within the maximum value and the minimum value of the variable range (i.e., the hatched range).

It should be noted that, if the timing of the charge sweep pulse is shifted to realize the desired electronic shutter speed, the faster the electronic shutter speed is, the larger the ratio of the variable portion to the entire light amount at that timing.

As described above, according to aspects of the invention, the image capturing system is provided with an illuminating unit configured to emit intermittent light that periodically illuminates an object, an image capturing element configured to capture an image of the object illuminated by the illuminating unit, and a control unit that controls the image capturing element to execute an image capturing operation. A period during which the object is periodically illuminated by the intermittent light includes an unstable starting period, a stable period and an unstable ending period, a timing at which the illuminating unit starts illuminating the object varying in the unstable starting period, a timing at which the illuminating unit stops illuminating the object varying in the unstable ending period. Further, the control unit controls the image capturing element to start and finish the image capturing operation within the stable period.

The present disclosure relates to the subject matter contained in Japanese Patent Application No. 2005-312618, filed on Oct. 27, 2005, which is expressly incorporated herein by reference in its entirety.

What is claimed is:

1. An image capturing system, comprising:
an illuminating unit configured to emit intermittent light that periodically illuminates an object;
an image capturing element configured to capture an image of the object illuminated by the illuminating unit; and
a control unit that controls the image capturing element to execute an image capturing operation, the image capturing operation including a start of exposure and a completion of accumulating electrical charges in the image capturing element,
wherein a period during which the object is periodically illuminated by the intermittent light includes an unstable starting period, a stable period and an unstable ending period, a timing at which the illuminating unit starts illuminating the object varying in the unstable starting period, a timing at which the illuminating unit stops illuminating the object varying in the unstable ending period, and
wherein the control unit controls the image capturing element to execute the image capturing operation only within the stable period.

2. The image capturing system according to claim 1, wherein the control unit transmits a sweep pulse to the image capturing element after the unstable start period has elapsed and a charge transfer pulse to the image capturing element before the unstable end period begins.

3. The image capturing system according to claim 2, wherein the control unit changes at least one of a timing at which the sweep pulse is transmitted to the image capturing element and a timing at which the charge transfer pulse is transmitted to the image capturing element to vary a charge accumulating period.

4. The image capturing system according to claim 3, wherein the time at which the sweep pulse is transmitted to the image capturing element is variable.

5. The image capturing system according to claim 3, wherein the time at which the charge transfer pulse is transmitted to the image capturing element is set to a timing immediately before the beginning of the unstable end period.

6. The image capturing system according to claim 3, wherein the illuminating unit is provided with:
a light source emitting continuous light; and
a rotational chopper mechanism having a rotatable member formed with a light shielding portion that shields the light emitted by the light source and a light transmitting portion that allows the light emitted by the light source to pass through.

7. The image capturing system according to claim 6, wherein the rotational chopper mechanism is provided with a motor which is controlled in accordance with a phase locked loop method using a signal synchronized with the charge transfer pulse.

8. An electronic endoscope system having an electronic endoscope and a processor, comprising:
an illuminating unit provided to the processor and is configured to emit intermittent light that is guided inside the electronic endoscope and emitted from a tip end of the electronic endoscope to periodically illuminate an object;
an image capturing element provided to the endoscope and is configured to capture an image of the object illuminated by the illuminating unit; and
a control unit provided to the endoscope and is configured to control the image capturing element to start and finish an image capturing operation, the image capturing operation including a start of exposure and a completion of accumulating electrical charges in the image capturing element,
wherein a period during which the object is periodically illuminated by the intermittent light includes an unstable starting period, a stable period and an unstable ending period, a timing at which the illuminating unit starts illuminating the object varying in the unstable starting period, a timing at which the illuminating unit stops illuminating the object varying in the unstable ending period, and
wherein the control unit controls the image capturing element to execute the image capturing operation only within the stable period.

9. The electronic endoscope system according to claim 8, wherein the control unit transmits a sweep pulse to the image capturing element after the unstable start period has elapsed and a charge transfer pulse to the image capturing element before the unstable end period begins.

10. The electronic endoscope system according to claim 9, wherein the control unit changes at least one of a timing at which the sweep pulse is transmitted to the image capturing element and a timing at which the charge transfer pulse is transmitted to the image capturing element to vary a charge accumulating period.

11. The electronic endoscope system according to claim 10, wherein the time at which the sweep pulse is transmitted to the image capturing element is variable.

12. The electronic endoscope system according to claim 10, wherein the time at which the charge transfer pulse is transmitted to the image capturing element is set to a timing immediately before the beginning of the unstable end period.

13. The electronic endoscope system according to claim 10, wherein the illuminating unit is provided with:
a light source emitting continuous light; and
a rotational chopper mechanism having a rotatable member formed with a light shielding portion that shields the light emitted by the light source and a light transmitting portion that allows the light emitted by the light source to pass through.

14. The electronic endoscope system according to claim 13, wherein the rotational chopper mechanism is provided with a motor which is controlled in accordance with a phase locked loop method using a signal synchronized with the charge transfer pulse.
